(19) United States
(12) Patent Application Publication
(10) Pub. No.: US 2004/0086891 A1
(43) Pub. Date: May 6, 2004

(54) Dehydrogenase oligomeric modulators

(76) Inventors: William G. Tatton, Yorktown Heights, NY (US); Katherine Borden, New Rochelle, NY (US)

Correspondence Address: LAHIVE & COCKFIELD, LLP., 28 STATE STREET, BOSTON, MA, 02109, US

(21) Appl. No.: 10/414,809

(22) Filed: Apr. 16, 2003

Related U.S. Application Data
(63) Continuation of application No. 09/327,200, filed on Jun. 7, 1999, now abandoned.
(60) Provisional application No. 60/088,771, filed on Jun. 10, 1998.
(60) Provisional application No. 60/092,054, filed on Jul. 8, 1998.

(51) Int. Cl. C12Q 1/68

(52) U.S. Cl. 435/6

(57) ABSTRACT

Methods of screening compounds which are oligomeric modulators of dehydrogenases such as glyceraldehyde-3-phosphate dehydrogenase (GAPDH), and such oligomeric modulators. In one aspect, methods are presented for identifying an oligomeric modulator of a dehydrogenase, comprising contacting the dehydrogenase with a compound under conditions which allow oligomerization of the dehydrogenase to occur; and measuring the ability of the compound to modulate oligomerization of the dehydrogenase as an indication of whether the compound is an oligomeric modulator of the dehydrogenase. In another aspect, methods are presented for identifying an oligomeric modulator of GAPDH, comprising contacting GAPDH with a compound under conditions which allow oligomerization of GAPDH to occur; and measuring the ability of the compound to modulate oligomerization of GAPDH as an indication of whether the compound is an oligomeric modulator of GAPDH. In another aspect, methods are presented for identifying antiapoptotic agents, comprising contacting GAPDH with a compound under conditions which allow oligomerization of GAPDH to occur; and measuring the ability of the compound to modulate oligomerization of GAPDH as an indication of whether the compound is an antiapoptotic agent.

### RELATED APPLICATIONS

This application claims the benefit of priority under 35 U.S.C. 119(e) to copending U.S. Provisional Application Nos. 60/088,771, filed on Jun. 10, 1998, and 60/092,054, filed on Jul. 8, 1998, the entire contents of both are incorporated herein by reference.

### BACKGROUND OF THE INVENTION

The progressive death of catecholaminergic neurons is central to the neurological deficits in Parkinson's disease (PD). Symptomatic therapy reduces PD neurological deficits but does not alter the progressive nature of the disease. To date, there is no firm evidence that the progression of PD can be slowed; therefore, the development of agents that slow disease progression is a major priority of PD research. Recent evidence suggests that apoptosis mediates neuronal death in PD. The explosion in the understanding of apoptosis and the application of that knowledge to PD has the potential to lead to interventions that could slow or block neuronal death in the disease.

Apoptosis can be initiated in neurons by many different forms of damage and proceeds in a step by step fashion with each step involving signaling by specific proteins. The signaling involves the cleavage, binding and inter-organelle movements of the proteins. The signaling pathways in the early phases of apoptosis depend on the form of damage that initiates the process. Accordingly, specific early pathways can be identified by changes in the levels and/or subcellular locations of specific proteins. The early signaling pathways converge onto a small number of effector signaling pathways that lead to the final degradative steps typical of apoptosis.

The dependence of apoptosis on cascades of signaling proteins opens the door to the interruption of the process and thereby to the rescue of damaged neurons. Apoptosis can be disrupted by changing the three dimensional conformation of apoptosis-promoting (pro-apoptotic) proteins, thereby blocking their function, or alternatively, by conformational changes that enhance the function of anti-apoptotic proteins that signal for interruption of specific steps in apoptotic pathways.

Apoptosis is a multi-step process that can be separated into three phases: initiation phase, effector phase and a final degradation phase (152). Events in the degradative phase of apoptosis, like nuclear chromatin condensation (NCC) and DNA cleavage, are commonly used to identify neurons in apoptosis. Electron microscopy (EM) of apoptotic cells reveals masses of condensed nuclear chromatin which fractionate into membrane-wrapped, chromatin-dense nuclear bodies. The cytoplasm also condenses with an intact organelle structure and buds into membrane wrapped fragments. These cell fragments are engulfed by macrophages without an inflammatory reaction. Gel electrophoresis generally demonstrates a 180-200 base pair “ladder” of endonuclease-digested DNA in apoptosis, although in some cases 300-500 base pair fragments occur (107, 109). In situ end labeling (ISEL) techniques attach a chromagen or fluorochrome to the cut 3′-OH ends of DNA to mark DNA cleavage (159). The NCC, typical of apoptosis, results from the stripping of proteins from DNA and condensation of the DNA. NCC can be visualized with fluorescent DNA binding dyes like bisbenzimide or YOYO-1 (159). Delineation of a subcellular structure is essential to the recognition of apoptosis so that the demonstration of appropriate changes in nuclear substructure coupled with the joint demonstration of DNA cleavage and NCC in the same nucleus provides irrefutable evidence for apoptotic nuclear degradation.

Changes in the expression of specific genes, and/or changes in the levels of their protein products, can serve as markers of different apoptotic pathways and apoptotic phases. The translocation of apoptosis-related proteins, concentrated in a specific subcellular organelle(s), to different organelles/compartments is fundamental to apoptosis signaling and can also serve to identify specific apoptotic pathways and phases.

Glyceraldehyde-3-phosphate dehydrogenase (GAPDH) catalyzes the conversion of glyceraldehyde-3-phosphate to 1,3-bisphosphoglycerate in the glycolytic pathway and converts NAD⁺ to the high energy carrier NADH. Other functions associated with GAPDH include the control of endocytosis, mRNA regulation, tRNA export, DNA replication, and DNA repair (135). GAPDH binds to the polyglutamine repeats in the abnormnal protein in Huntington's disease (20). GAPDH mRNA and protein levels increase during apoptosis of cultured cerebellar neurons (59, 138). Antisense oligonucleotides against GAPDH mRNA decrease cerebellar neuronal apoptosis (59). Koningic acid inhibits GAPDH glycolytic function and induces apoptosis in NG108-15 cells (103). It binds to the Rossman fold of the protein, where NAD⁺ is converted to NADH and where AU-rich RNA binds to GAPDH.

Western blots have suggested a shift of GAPDH protein from the cytosolic to the nuclear fraction in cerebellar cells (121), thymocytes, PC12 cells and cultured cortical neurons entering apoptosis (122). GAPDH upregulation and nuclear translocation appears to occur during the initiation phase or early effector phase in neuronally differentiated PC12 cells and fibroblasts. GAPDH nuclear translocation has been found in melanin-containing neurons in the PD SNc, and, accordingly, a GAPDH-dependent apoptosis may contribute to dopaminergic cell death in PD. The role of GAPDH in apoptosis has not been completely understood.

### SUMMARY OF THE INVENTION

The present invention is based, at least in part, on the discovery that molecules or compounds that interfere with GAPDH's ability to form a tetramer are antiapoptotic agents.

The present disclosure relates to methods of screening compounds which are oligomeric modulators of dehydrogenases, e.g., lactate dehydrogenase (LADH) and glyceraldehyde-3-phosphate dehydrogenase (GAPDH), as well as identifying such dehydrogenase oligomeric modulators (DOMs). In one aspect, methods are presented for identifying an oligomeric modulator of a dehydrogenase. The method involves contacting the dehydrogenase with a compound under conditions which allow oligomerization of the dehydrogenase to occur; and measuring the ability of the compound to modulate oligomerization of the dehydrogenase as an indication of whether the compound is a DOM. In an embodiment, the ability of the compound to modulate oligomerization of the dehydrogenase is its ability to modulate the level of oligomerization of the dehydrogenase.

In one aspect, methods are presented for identifying an oligomeric modulator of GAPDH. The method involves contacting GAPDH with a compound under conditions which allow oligomerization of GAPDH to occur; and measuring the ability of the compound to modulate oligomerization of GAPDH as an indication of whether the compound is an oligomeric modulator of GAPDH. In an embodiment, the ability of the compound to modulate oligomerization of GAPDH is its ability to prevent the formation of a tetramer of GAPDH. In a further embodiment, the ability of the compound to modulate oligomerization of GAPDH is its ability to enhance the formation of a dimer of GAPDH.

In another aspect, methods are presented for identifying antiapoptotic agents, comprising contacting GAPDH with a compound under conditions which allow oligomerization of GAPDH to occur; and measuring the ability of the compound to modulate oligomerization of GAPDH as an indication of whether the compound is an antiapoptotic agent. In an embodiment, the ability of the compound to modulate oligomerization of GAPDH is its ability to prevent the formation of a tetramer of GAPDH. In a further embodiment, the ability of the compound to modulate oligomerization of GAPDH is its ability to enhance the formation of a dimer of GAPDH. In yet another embodiment, the ability of the compound to modulate oligomerization of GAPDH is its ability bind to GAPDH and thus prevent GAPDH oligomer formation.

In another embodiment, methods are disclosed for screening a library of compounds which are oligomeric modulators of dehydrogenases, e.g., lactate dehydrogenase (LADH) and glyceraldehyde-3-phosphate dehydrogenase (GAPDH), as well as identifying such dehydrogenase oligomeric modulators (DOMs). In one aspect, methods are presented for identifying DOMs from a library of compounds. The method involves contacting the dehydrogenase with a sample of a library of compounds under conditions which allow oligomerization of the dehydrogenase to occur; classifying any oligomers formed; and identifying the compounds that modulate oligomerization of the dehydrogenase as an indication of whether the compound(s) is an oligomeric modulator of the dehydrogenase. In an embodiment, the ability of the compound(s) to modulate oligomerization of the dehydrogenase is its ability to prevent the formation of a oligomer of the dehydrogenase.

In another embodiment, methods are disclosed for screening a library of compounds for their ability to modulate oligomerization of GAPDH, comprising contacting GAPDH with a sample of a library of compounds under conditions which allow oligomerization of GAPDH to occur; classifying any oligomers formed; and identifying the compounds that modulate oligomerization of GAPDH as an indication of whether the compound is an oligomeric modulator of GAPDH. In an embodiment, the ability of the compound(s) to modulate oligomerization of GAPDH is its ability to prevent the formation of a tetramer of GAPDH. In a further embodiment, the ability of the compound(s) to modulate oligomerization of GAPDH is its ability to enhance the formation of a dimer of GAPDH. In yet another embodiment, the ability of the compound(s) to modulate oligomerization of GAPDH is its ability bind to GAPDH and thus prevent GAPDH oligomer formation.

In certain embodiments, the compound to be tested is derived from a library of small molecules. In another embodiment, the ability of the compound to modulate oligomerization of GAPDH is its ability bind to GAPDH and thus prevent GAPDH oligomer formation can be predicted through use of molecular modeling. In another embodiment, the compound is a naturally occurring small organic molecule. In a preferred embodiment, the compound may be, e.g., dibenzo[bf]oxepin-10-ylmethyl-prop-2-ynyl-amine (CGP3466), or (−)-desmethyldeprenyl.

### BRIEF DESCRIPTION OF THE DRAWINGS

FIG. 1 illustrates the binding of an analog of desmethyl deprenyl, CGP 3466, to the tetramer form of GAPDH;

FIG. 2 illustrates another view of the binding of CGP 3466 to the tetramer form of GAPDH; and

FIG. 3 is size exclusion chromatographic profiles showing the effect of CGP 3466 and (−)-desmethyldeprenyl to on the oligomeric state of GAPDH.

### DETAILED DESCRIPTION OF THE INVENTION

The present disclosure relates to methods of screening compounds which are oligomeric modulators of dehydrogenases, in particular glyceraldehyde-3-phosphate dehydrogenase (GAPDH), as well as identifying such GAPDH oligomeric modulators. For example, certain compounds that bind to GAPDH that decrease the capacity of GAPDH to form tetramers have been identified. The shift from a tetrameric to a dimeric conformation reduces the proapoptotic capacity of GAPDH, since it is associated with a decrease in some forms of neuronal apoptosis.

Before further description of the invention, certain terms employed in the specification, examples and appended claims are, for convenience, collected here.

Definitions

As used herein the term “compound” includes any reagent which is employed in the methods of the disclosure and assayed for its utility as an oligomeric modulator of a dehydrogenase, e.g., GAPDH, by the ability of the compound to prevent or inhibit the formation of an oligomer of the dehydrogenase, e.g., tetramer of GAPDH. Exemplary compounds which can be screened for activity include, but are not limited to, peptides, non-peptidic compounds, nucleic acids, carbohydrates, small organic molecules, and natural product extract libraries. The term “non-peptidic compound” is intended to encompass compounds that are comprised, at least in part, of molecular structures different from naturally-occurring L-amino acid residues linked by natural peptide bonds. However, “non-peptidic compounds” are intended to include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic compounds” also are intended to include natural products.

“Antiapoptotic agent” is intended to include compounds or compositions which ameliorate, modulate or prevent apoptosis.

“Apoptosis” is intended to include the process in which catabolic enzymes degrade essential macromolecules leading to a characteristic biochemical and ultra-structural death phenoptype. In apoptosis, cellular fragments are membrane wrapped and are removed by phagocytes without evidence of inflammation. (see also 152, appended hereto and incorporated herein by reference.) In sharp contrast, necrosis involves extrusion of cellular contents which induces a easily detectable inflammatory response.

“Dehydrogenase” is intended to include enzymes which catalyze oxidation by the removal of hydrogen, e.g., lactate dehydrogenase (LADH), glyceraldehyde-3-phosphate dehydrogenase (GAPDH), etc.

“Dehydrogenase oligomeric modulator” is intended to include those compounds or compositions which prevent, inhibit or interfere with dehydrogenase oligomer formation, e.g., tetramer of GAPDH, wherein “oligomer” preferably refers to more than two, more preferably more than three, and even more preferably more than four, monomeric subunits. Also included are those compounds which enhance or promote the formation of a lower form of the dehydrogenase, e.g., a dimer.

“Conditions which allow oligomerization to occur” is intended to include in vivo or in vitro conditions favorable to the formation of the oligomeric form of a dehydrogenase.

“Measuring the ability of a compound to modulate oligomerization” is intended to include measuring the ability of a compound to prevent the formation of a dehydrogenase oligomer, e.g., tetramer, and the ability of a compound to enhance or promote the formation of a lower form of the dehydrogenase, e.g., a dimer.

“Oligomeric modulation” is intended to include the prevention or inhibition of, or interference with dehydrogenase oligomer, e.g., tetramer, formation. Also included is the enhancement or promotion of the formation of a lower form of the dehydrogenase, e.g., a dimer.

GAPDH induces apoptosis in cultured neurons. It has been shown that GAPDH densely translocates to the nucleus early in apoptosis in cultured neurons and preliminary studies have shown GAPDH nuclear translocation in melanin-containing neurons in the PD SNc. Based on studies involving the binding of antiapoptotic agents to GAPDH, it is believed that the tetrameric form of GAPDH is essential to apoptosis induction, and in particular, that conversion of GAPDH from a tetramer to a dimer prevents it from inducing apoptosis.

A non-limiting step by step model for GAPDH induction of apoptosis is presented: 1) NAD⁺ levels modulate the participation of GAPDH in glycolysis, such that the higher the energy demands of the cell, as signaled by NAD⁺ levels, the greater the amounts of GAPDH avaiiable for glycolysis; 2) major stress or toxic exposure will decrease the conversion of NAD⁺ to NADH so that high NAD⁺ levels will increase freed GAPDH; 3) similarly, high levels of superoxide radicals, nitric oxide or peroxynitrite free GAPDH from AU-RNA; 5) freed GAPDH will enter the nucleus and bind to DNA or DNA-associated proteins; 6) the nuclear translocation of GAPDH signals or facilitates apoptosis; 7) the induction by apoptosis by GAPDH requires the protein to be in a tetrameric form; and 8) compounds which may be identified by the methods disclosed herein, such as DES or CGP3466, which tend to maintain GAPDH as a dimer, reduce the induction of apoptosis by GAPDH. Note that this model is not meant to be a limitation on the claimed invention, but rather one possible explanation of the biochemical mechanisms involved.

Methods of Identifying Compounds which Modulate Oligomerization of GAPDH

In an embodiment, the invention provides for methods for identifying an compound which modulates oligomerization of a dehydrogenase, e.g., GAPDH. The methods involve contacting the dehydrogenase with a compound under conditions which allow oligomerization of the dehydrogenase to occur; and measuring the ability of the compound to modulate oligomerization of the dehydrogenase as an indication of whether the compound is an oligomeric modulator of the dehydrogenase. The methods, in ah embodiment, further involve contacting GAPDH with a compound under conditions which allow oligomerization of GAPDH to occur; and measuring the ability of the compound to modulate oligomerization of GAPDH as an indication of whether the compound is an oligomeric modulator of GAPDH. In an embodiment, the ability of the compound to modulate oligomerization of GAPDH is its ability to prevent the formation of a tetramer of GAPDH. In a further embodiment, the ability of the compound to modulate oligomerization of GAPDH is its ability to enhance the formation of a dimer of GAPDH.

Without wishing to be bound by theory, it is believed that binding of compounds to GAPDH effectively prevents oligomer, e.g., tetramer, formation and enhances dimer formation, resulting in an amelioration or prevention of apoptosis.

A variety of different techniques can be used to determine whether a compound modulates oligomerization of dehydrogenases such as GAPDH. One technique employs molecular modeling of the oligomeric/multimeric protein structure to determine compounds which will bind to the channel(s) formed where the monomers meet and thus inhibit or prevent oligomer formation, such as is set forth in non-limiting detail in Example 2. This method may also be used to screen libraries of compounds, using techniques familiar to those of ordinary skill in the art to determine compounds in the libraries which are GAPDH oligomeric modulators and are likely to also be an antiapoptotic agent.

Another technique, which can also be used to confirm the results of a molecular modeling study, utilizes size exclusion chromatography (SEC) to classify the oligomers formed in the contacting step. For example, if the elution profile indicates that the primary product formed in the contacting step is not a tetramer, e.g., monomer or dimer, then the compound is a GAPDH oligomeric modulator and is likely to also be an antiapoptotic agent, consistent with the hypothetical model discussed above. Such methods may also be applied, as described herein, to screen libraries of compounds using techniques familiar to those of ordinary skill in the art to determine compounds in the libraries which are GAPDH oligomeric modulators and are likely to also be an. antiapoptotic agent. One example of assessing the ability of a compound to bind to GAPDH and thus modulate GAPDH oligomerization is set forth in non-limiting detail in Example 3, below.

Test Compounds

Compounds for testing in the present methods can be derived from a variety of different sources. In preferred embodiments, libraries of compounds are tested in the present methods to identify compounds which modulate oligomerization of GAPDH.

A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al. 1992. J. Am. Chem. Soc. 114:10987; DeWitt et al. 1993. Proc. Nail Acad. Sci. USA 90:6909) peptoids (Zuckermann. 1994. J. Med. Chem. 37:2678) oligocarbamates (Cho et al. 1993. Science 261:1303), and hydantoins (DeWitt et al. supra). Rebek et al. have described an approach for the synthesis of molecular libraries of small organic molecules with a diversity of 104-105 (Carell et al. 1994. Angew. Chem. Int. Ed. Engl. 33:2059; Carell et al. Angew. Chem. Int. Ed. Engl. 1994. 33:2061).

Candidate compound librariess can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. Anticancer Drug Des. 1997. 12:145).

Exemplary compounds which can be screened for activity include, but are not limited to, peptides, nucleic acids e.g., small oligonucleotides, carbohydrates, small organic molecules, and natural product extract libraries.

Other exemplary methods for the synthesis of molecular libraries can be found in the art, for example in: Erb et al. 1994. Proc. Natl. Acad. Sci. USA 91:11422; Horwell et al. 1996 Immunopharmacology 33:68; and in Gallop et al. 1994. J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990)Science 249:404-406); (Cwirla et al. (1990) Proc. Natl. Acad. Sci. 87:6378-6382); (Felici (1991)J. Mol. Biol. 222:301-310); (Ladnersupra.). Other types of peptide libraries may also be expressed, see, for example, U.S. Pat. Nos. 5,270,181 and 5,292,646). In still another embodiment, combinatorial polypeptides can be produced from a cDNA library.

Formulations Comprising Compounds Identified in the Present Assays

The invention provides pharmaceutically acceptable compositions which include a therapeutically-effective amount or dose of a compound identified in any of the instant assays and one or more pharmaceutically acceptable carriers (additives) and/or diluents.

As described in detail below, the pharmaceutical compositions can be formulated for administration in solid or liquid form, including those adapted for the following: (1) oral administration, for example, aqueous or non-aqueous solutions or suspensions, tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream, foam, or suppository; or (5) aerosol, for example, as an aqueous aerosol, liposomal preparation or solid particles containing the compound.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the compounds of the invention from one organ, or portion of the body, to another organ, or portion of the body without affecting its biological effect. Each carrier should be “acceptable” in the sense of being compatible with other ingredients of the composition and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microbes may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions may be administered to epithelial surfaces of the body orally, parenterally, topically, rectally, nasally, intravaginally, intracisternally. They are of course given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a sucrose octasulfate and/or an antibacterial, drug or other material other than directly into the central nervous system, such that it enters the subject's system and, thus, is subject to metabolism and other like processes, for example, subcutaneous administration.

In some methods, the compositions can be topically administered to any epithelial surface. An “epithelial surface” according to this invention is defined as an area of tissue that covers external surfaces of a body, or which lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, douches, drops, troches, chewing gums, lozenges, mouthwashes, rinses.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinypyrrolidone, silica and derivatives thereof, xanthan gum, kaolin, talc, starch and derivatives thereof, paraffin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate, and which is solid at room temperature, but liquid at body temperature and, therefore, will melt in the rectum or vaginal cavity and release the active agent.

Compositions which are suitable for vaginal administration also include pessaries, tampons, creams, gels, pastes, (foams, films, or spray compositions containing such carriers as are known in the art to be appropriate. The carrier employed in the sucrose octasulfate/contraceptive agent should be compatible with vaginal administration and/or coating of contraceptive devices. Combinations can be in solid, semi-solid and liquid dosage forms, such as diaphragm, jelly, douches, foams, films, ointments, creams, balms, gels, salves, pastes, slurries, vaginal suppositories, sexual lubricants, and coatings for devices, such as condoms, contraceptive sponges, cervical caps and diaphragms.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolatum. Exemplary ophthalmic compositions include eye ointments, powders, solutions and the like.

Powders and sprays can contain, in addition to sucrose octasulfate and/or antibiotic or contraceptive agent(s), carriers such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), proteins like serum albumin, sorbitan esters, oleic acid, lecithin, amino acids such as glycine, buffers, salts, sugars or sugar alcohols. Aerosols generally are prepared from isotonic solutions.

Compositions can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the antiinfective agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the compositions of this invention can be aqueous or oleaginous suspension. These suspensions may be formulated according to techniques known in the art using suitable dispersing or wetting agents and suspending agents. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The invention is further illustrated by the following examples which should not be construed as further limiting the subject invention. The contents of all references, issued patents, and published patent applications cited throughout this application including the background are hereby incorporated by reference.

### EXAMPLE 1

Binding of DOMs to a Dehydrogenase and Increase of Dehydrogenase Levels in Early Apoptosis

It was shown that (−)-desmethyldeprenyl and CGP3466 bind to GAPDH and prevent the nuclear relocation of GAPDH. It was also shown that there was an increase in GAPDH levels found in early apoptosis. Photoaffinity binding techniques and a BODIPY chromogen attached to dibenzo[bf]oxepin-10-ylmethyl-prop-2-ynyl-amine (CGP3466), were used to study the protein and subcellular binding of (−)-desmethyldeprenyl (DES) and CGP3466. These studies showed CGP3466 bound to four proteins: tubulin, actin, synapsin and GAPDH.

The subcellular localization of GAPDH in PC12 cells entering apoptosis using immunocytochemistry and LCSM has been studied. In early apoptosis (at 3-12 hours after serum and NGF withdrawal GAPDH immunodensity had decreased in the cytosol, but had become densely concentrated in the nucleus. CGP3466 or DES treatment blocked the nuclear translocation GAPDH immunodensity. Similarly, treatment of partially differentiated PC12 cells or fibroblasts with the nitric oxide (NO) donor SNAP induced apoptosis and similar nuclear translocation of GAPDH. NO enters the Rossman fold of GAPDH and frees the enzyme from AU-RNA (60, 90, 170). Treatment of fibroblasts with NAD⁺ induced the dense translocation of GAPDH to the nucleus. NAD⁺ frees GAPDH from AU-RNA while the conversion of NAD⁺ to NADH allows this association to be re-established (40, 86, 102, 169).

GAPDH cDNA was cloned from rat brain using oligonucleotides designed to the 5′- and 3′-ends of the coding region of rat GAPDH with PCR. The cDNA was used to produce a GAPDH-green fluorescent protein (GFP) construct by adding a 3′ oligo (Gly)s to the C-terminus of GAPDH followed by a BamHI site to allow subcloning, in frame, into pEGFP-1 (Clontech). The resulting construct contains GAPDH followed by (Gly)5 and enhanced GFP. The insert was subcloned into the Hindlil-Notl sites of pcDNA3. The GAPDH-GFP construct has been transiently transfected into COS1 cells and stably transfected into HEK 293 cells.

Apoptosis was induced in the living COS and Hek cells maintained in a physiological chamber on LCSM. Living cells were shown to have little or no GAPDH-GFP protein in their nuclei and showed a cytosolic distribution for the fusion protein that seemed identical to that shown with immunocytochemistry for GAPDH. The GAPDH-GFP fusion protein was shown to concentrate in the nucleus of a proportion of the cells in the first two hours after exposure to apoptosis initiating agents like Rose Bengal. These experiments established that GAPDH nuclear translocation occurs dynamically during the very early stages of apoptosis and indicate that GAPDLH participates in the initiation phase of apoptosis.

As well as the nuclear translocation, GAPDH levels increase rapidly in the early phases of apoptosis. The protein levels begin to increase at 1.5 to 2.0 hours after trophic withdrawal and that DES, CGP3466 and NAC prevent the increase. At least three molecules associated with toxin-induced neuronal apoptosis—nitric oxide, superoxide radical and peroxynitrite—have the capacity to free GAPDH from AU-rich RNA (Beckman and Koppenol, 1996). Nitric oxide modifies the structure of GAPDH and appears to free it from AU-rich RNA (Brune and Lapetina, 1995; Brune and Lapetina, 1996; Itoga et al., 1997; McDonald and Moss, 1994). Oxygen free radicals enhance the binding of NAD⁺ to GAPDH (Marin et al., 1995). If, in fact, GAPDH translocation to the nucleus does initiate or facilitate apoptosis, then ROS and NO may induce apoptosis through GAPDH. We also found that NAD⁺ releases GAPDH from AU-rich RNA and thereby, increases the enzyme's glycolytic activity.

### EXAMPLE 2

Modeling of a Dehydrogenase

Materials and Methods:

Crystal structure coordinates from the Brookhaven data base were used to produce GAPDH models. To date, there is not a protein structure of GAPDH from a rat source so the rat amino acid sequence was built onto the GAPDH structure of another organism using the molecular modeling program INSIGHT (Biosym). There is high conservation between GAPDH structures obtained from different sources and organisms leading to the belief that the rat GAPDH structure should be very similar to that from other animals. Several drug compounds were constructed, including N-acetyl cysteine, desmethyl deprenyl, and CGP3466 using INSIGHT.

Finding the Binding Site of CGP3466:

Inspection of the GAPDH model indicated that there is a large channel formed where the four monomers meet to form GAPDH. As the active site cleft was too large to consider that CGP3466 or desmethyl deprenyl bound there this channel was investigated. Any compounds which bind the active site cleft would be expected to decrease glycolysis and RNA binding which also occurs at this site. Both CGP 3466 and desmethyl deprenyl fit in the channel. The specific respective binding sites are not known, but molecular modeling data suggested that inserting drugs at the interface between the four monomers could both act to either stabilize the tetramer or cause it to fall apart. Both of the above-mentioned drugs disrupt the tetramer forming dimers. Also, if a destablizing agent such as sodium dodecyl sulfate is added, untreated protein becomes monomeric but treated protein remains dimeric. It was noted that the dimer comprises a “mini-channel” in which the drugs can bind.

Confirming the Location of the Binding Site:

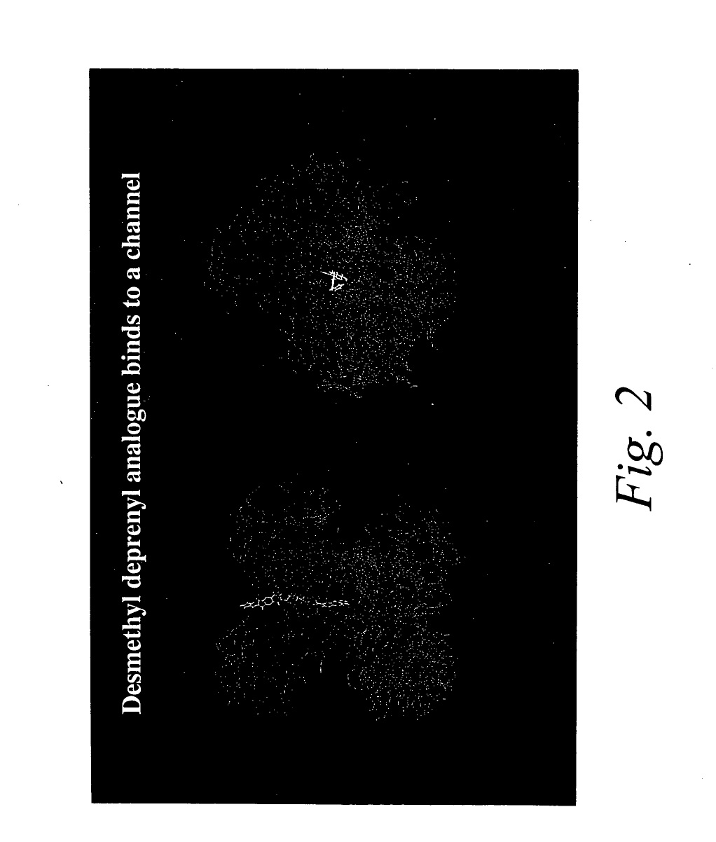

Antibody blocking experiments were conducted to confirm that CGP3466 bound GAPDH in the channel. In FIG. 2, GAPDH is shown in blue with the site for an antibody which was raised to a certain part of GAPDH is green. If one first treats the protein with antibody then drug there is no binding, indicating that blocking the channel inhibits binding. Other antibodies to GAPDH do not block drug binding.

Selection of Drug Targets:

It appears likely that desmethyl deprenyl and CGP3466 could bind other members of the dehydrogenase family. Most of these proteins are oligomeric/multimeric and channels are formed where these monomers meet. Further, these proteins contain a NAD binding site known as the Rossmann fold. This combination of common structural features means that the channel size would be similar for most dehydrogenases. For instance, molecular modeling of lactate dehydrogenase (LADH), a hexamer, suggest that both CGP3466 and desmethyl deprenyl bind in the LADH channel.

### EXAMPLE 3

Identifying a Dehydrogenase Oligomeric Modulator

A dehydrogenase oligomeric modulator is identified using size exclusion chromatographic (SEC) techniques. A test compound is tested for its ability to prevent tetramer formation of GAPDH and compounds capable of preventing or inhibiting such formation are identified as DOMs. SEC is used to determine if potential compounds affect the oligomeric state of GAPDH (or any oligomeric protein) when contacted with GAPDH under conditions which promote oligomer formation. This method can be further refined and sped up (i.e., faster data collection) by using SEC-high performance liquid chromatographic (SEC-HPLC) methods. An example is set forth below.

Column Formation

A column was made using a disposable 1 cm diameter column (BIORAD). The chosen matrix for the column was Sephacryl H-300 (PHARMACIA). The column was poured by mixing the beads (SEPHACRYL) in a buffer and layering into the column until the bed was about 12 cm long. The buffer used was 50 mM TRIS—HCl, pH 8.0. The column was then washed with the same buffer for 10 column volumes.

Column Standardization/Calibration

A selection.of protein standards of known molecular weight was run through the column. Hence, the elution time any of the proteins can be calculated by collecting fractions of equal volume (500 ml) and measuring for protein by optical density at 280 nm.

Once the column is calibrated, the molecular weight of the eluted fractions (in this case, GAPDH monomers, dimers and tetramers) may be determined. Hence, one can determine from an elution profile 1) the relative proportion of GAPDH monomer, dimer and tetramer, and 2) any binding of GAPDH to other molecules such as DNA or RNA (which is believed to decrease upon contact of GAPDH with compounds which interfere with tetramer formation/promote dimer formation.

Experimentation

Prior to moving the proteins to the column they are contacted with candidate compound and mixed at room temperature in, e.g., nanomolar concentrations under conditions known to those skilled in the art.

The mixture prepared above is then added to the column and eluted with 50 mM TRIS—HCl pH 8.0 buffer. 500 μl fractions of the eluent are collected. The optical density of the fractions is then determined, e.g., at 280 nm, and an elution profile prepared and analyzed to determine the extent of oligomerization.

### EXAMPLE 4

Identification of (−)-Desmethyldeprenyl and CGP3466 as Oligomeric Modulators of GAPDH

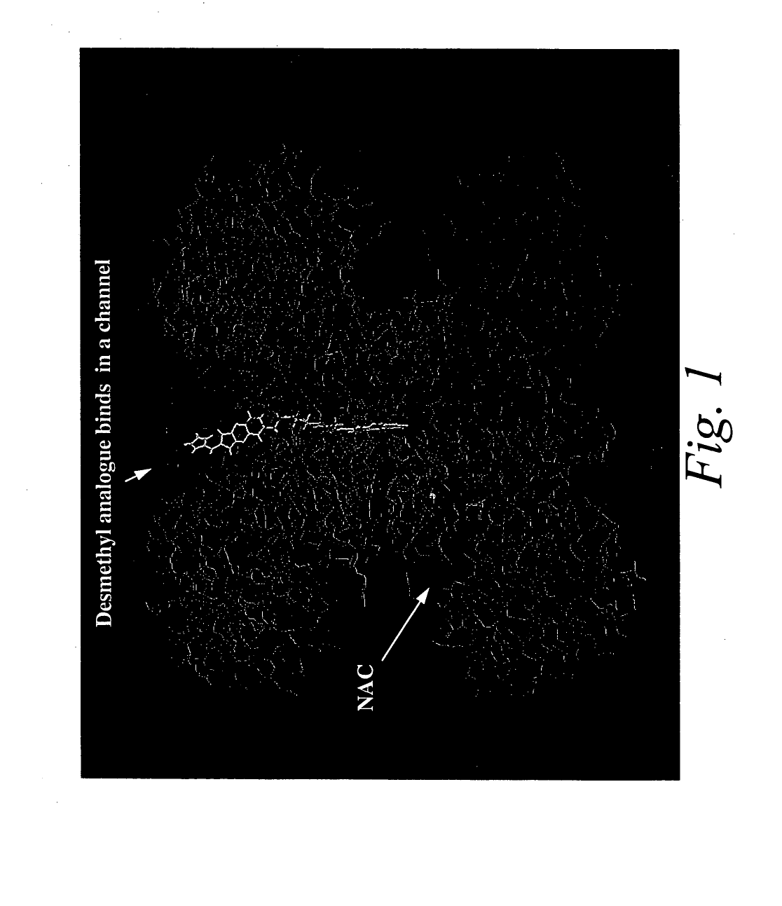

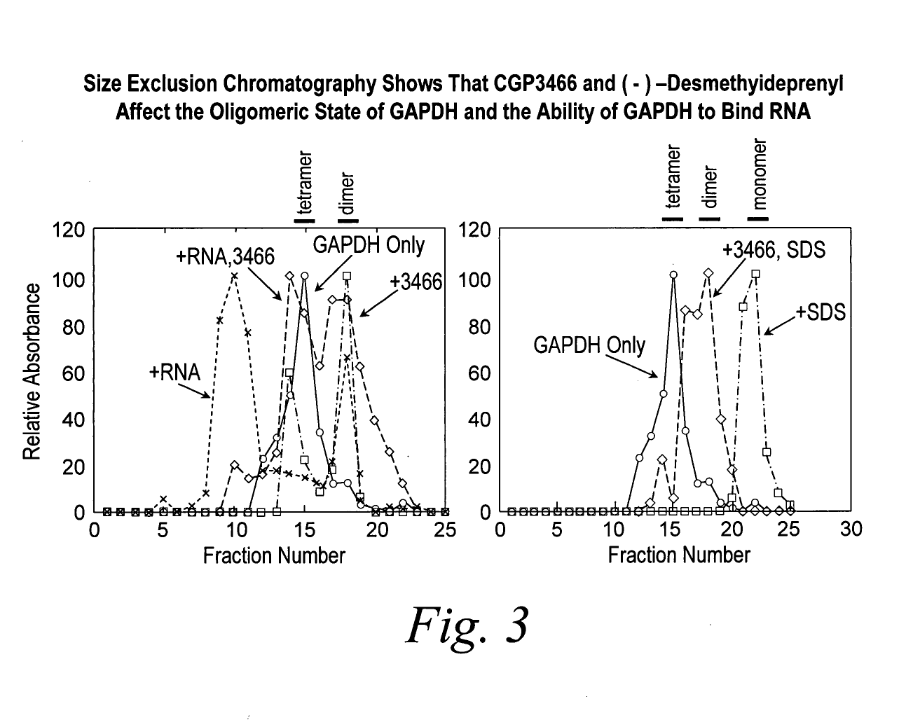

GAPDH is a tetramer consisting of four identical monomers (FIG. 1). The tetramer exists in equilibrium between tetramers, dimers and monomers. This equilibrium strongly favors the tetrameric form. Upon tetramer formation, there is a channel present between the four monomers. Size exclusion chromatography as in Example 3 was used to show that GAPDH is largely in a tetrameric form when bound to poly-U RNA, but is converted to a dimer in the presence of certain compounds, e.g., DES or CGP3466 (FIG. 3). Alternatively, GAPDH takes a monomeric form in SDS but certain compounds, e.g., DES or CGP3466, convert it to a dimer. As such, the ability of (−)-desmethyldeprenyl and CGP3466 to decrease GAPDH tetramer and increase GAPDH dimer in vitro identifies these compounds as oligomeric modulators of GAPDH.

Equivalents

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

Literature Cited

1. Agard, D A, and Sedat, J W. 1983. Three-dimensional architecture of a polytene nucleus. Nature 302:676-681.

2. Agid, Y. 1995. Aging, disease and nerve cell death. Bull Acad Natl Med 179:1193-203.

3. Anglade, P, Vyas, S, JavoyAgid, F, Herrero, M T, Michel, P P, Marquez, J, MouattPrigent, A, Ruberg, M, Hirsch, E C, and Agid, Y. 1997. Apoptosis and autophagy in nigral neurons of patients with Parkinson's disease. Histology and Histopathology 12:25-31.

4. Ansari, K S, Yu, P H, Kruck, T P, and Tatton, W G. 1993. Rescue of axotomized immature rat facial motoneurons by R(−)-deprenyl: stereospecificity and independence from monoamine oxidase inhibition. J. Neurosci 13:4042-53.

5. Armstrong, R C, Aja, T J, Hoangi K D, Gaur, S, Bai, X, Alnemri, E S, Litwack, G, Karanewsky, D S, Fritz, L C, and Tomaselli, K J. 1997. Activation of the CED3/1CE-related protease CPP32 in cerebellar granule neurons undergoing apoptosis but not necrosis. Journal of Neuroscience 17:553-562.

6. Attardi, G, Yoneda, M, and Chomyn, A. 1995. Complementation and segregation behavior of disease causing mitochondrial DNA mutations in cellular model systems. Biochim BiophysActa 1271:241-8.

7. Banati, R B, Daniel, S E, and Blunt, S B. 1998. Glial pathology but absence of apoptotic nigral neurons in long-standing Parkinson's disease [In Process Citation]. MovDisord 13:221-7.

8. Bandmann, O, Sweeney, M G, Daniel, S E, Marsden, C D, and Wood, N W. 1997. Mitochondrial DNA polymorphisms in pathologically proven Parkinson's disease. Journal of Neurology 244:262-265.

9. Beham, A, Marin, M C, Fernandez, A, Herrmann, J, Brisbay, S, Tari, A M, LopezBerestein, G, Lozano, G, Sarkiss, M, and McDonnell, T J. 1997. Bci-2 inhibits p53 nuclear import following DNA damage. Oncogene 15:2767-2772.

10. Bellamy, C O. 1997. p53 and Apoptosis. Br. Med. Bull. 53:522-538.

11. Benecke, R, Strumper, P, and Weiss, H. 1993. Electron transfer complexes I and IV of platelets are abnormal in Parkinson's disease but normal in Parkinson-plus syndromes. Brain 116:1451-63.

12. Bernardi, P, Broekemeier, K M, and Pfeiffer, D R. 1994. Recent progress on regulation of the mitochondrial permeability transition pore; A cyclosporin-sensitive pore in the inner mitochondrial membrane. J. Bioenerget. Biomemb. 26:509-517.

13. Bodnar, A G, Cooper, J M, Holt, I J, Leonard, J V, and Schapira, A H. 1993. Nuclear complementation restores mtDNA levels in cultured cells from a patient with mtDNA depletion. Am J Hum Genet 53:663-9.

14. Borden, K L B, Boddy, M N, Lally, J, O'Reilly, N J, Martin, S, Howe, K, Solomon, E, and Freemont, P S. 1995. The solution structure of the RING finger domain from the Acute Promyelocytic Leukaemia protoonocprotein, pml. EMBO J. 14:1532-1541.

15. Borden, K L B, CampbellDwyer, E J, and Salvato, M S. 1997. The promyelocytic leukemia protein PML has a pro-apoptotic acitvity mediated through its RING. Febs. Leff 418.

16. Borden, K L B, Campbell-Dwyer, E J, and Salvato, M S. 1988. An arenavirus RING (zinc-binding) protein binds the oncoprotein PML and relocates PML nuclear bodies to the cytoplasm. J. Virol. 72:758-766.

17. Borden, K L B, Lally, J M, Martin, S R, O'Reilly, N J, Solomon, E, and Freemont, P S. 1996. In vivo and in vitro characterisation of the B1 and B2 zinc-binding domains from the acute promyelocytic leukemia protein PML. Proc. Natl. Acad. Sci. U.S.A 93:1601-1606.

18. Brown, J, Higo, H, McKalip, A, and Herman, B. 1997. Human papillomavirus (HPV) 16 E6 sensitizes cells to atractyloside-induced apoptosis: role of p53, ICE-like proteases and the mitochondrial permeability transition. J. Cell Biochem 66:245-55.

19. Brown, M D, Shoffner, J M, Kim, Y L, Jun, A S, Graham, B H, Cabell, M F, Gurley, D S, and Wallace, D C. 1996. Mitochondrial DNA sequence analysis of four Alzheimer's and Parkinson's disease patients. American Journal of Medical Genetics 61:283-289.

20. Burke, J R, Enghild, J J, Martin, M E, Jou, Y, Myers, R M, Roses, A D, Vance, J M, and Strittmatter, W J. 1996. Huntingtin and DRPLA proteins selectively interact with the enzyme GAPDH. Nature Medicine 2:347-350.

21. Caeiles, C, Heimberg, A, and Kann, M. 1994. p53-dependent apoptosis in the absence of p53-target genes. Nature 370:220-223.

22. Carayon, P, Portier, M, Dussossoy, D, Bord, A, Petipretre, G, Canat, X, Le Fur, G, and Casellas, P. 1996. Involvement of peripheral benzodiazepine receptors in the protection of hematopoletic cells against oxygen radical damage. Blood 87:3170-3178.

23. Carri, M T, Ferri, A, Battistoni, A, Famhy, L, Gabbianelli, R, Poccia, F, and Rotilio, G. 1997. Expression of a Cu,Zn superoxide dismutase typical of familial amyotrophic lateral sclerosis induces mitochondrial alteration and increase of cytosolic Ca2+ concentration in transfected neuroblastoma SH-SY5Y cells. FEBS Letters 414:365-368.

24. Chacon, E, Reece, J M, Nieminen, A L, Zahrebelski, G, Herman, B, and Lemasters, J J. 1994. Distrubtion of electrical potential, pH, free Ca2+, and volume inside cultured adult rabbit cardiac myocytes during chemical hypoxia: A multiparameter digitized confocal microscopic study. Biophys 66:942-952.

25. Chalmers, R M E, and Fine, A. 1991. The influence of L-dopa ofsurvival and outgrowth of fetal mesencephalic dopaminergic neurons in vitro and after intracerebral transplantation. In: Intracerebral transplantation in movement disorders. Lindvall et al. Elsevier, London pp 333-342.

26. Chalmers-Redman, R M, Priestley, T, Kemp, J A, and Fine, A. 1997. In vitro propagation and inducible differentiation of multipotential progenitor cells from human fetal brain. Neuroscience 76:1121-8.

27. Charriaut-Mariangue, C, and Ben-Ari, Y. 1995. A cautionary note on the use of the TUNEL stain to determine apoptosis. Neuro Report 7:61-64.

28. Chauchan, D, Pandey, P, Ogata, A, Teoh, G, Krett, N, Haigren, R, Rosen, S, Kufe, D, Kharbanda, S, and Anderseon, K. 1997. Cytochrome c-dependent and -independent induction of apoptosis in multiple myeloma cells. J. Biol. Chem. 272:29995-29997.

29. Chernyak, B V, and Bernardi, P. 1996. The mitochondrial permeability pore is modulated by oxidative agents through both pyridine nucleotides and glutathione at two separate sites. Eur. J. Biochem. 238:623-630.

30. Chomyn, A, Lai, S T, Shakeley, R, Bresolin, N, Scarlato, G, and Attardi, G. 1994. Platelet-mediated transformation of mtDNA-less human cells: analysis of phenotypic variability among clones from normal individuals—and complementation behavior of the tRNALys mutation causing myoclonic epilepsy and ragged red fibers. Am J Hum Genet 54:966-74.

31. Cohen, G M. 1997. Caspases: the executioners of apoptosis. Biochemical Journal 326:1-16.

32. Davey, G P, and Clark, J B. 1996. Threshold effects and control of oxidative phosphorylation in nonsynaptic rat brain mitochondria. Journal of Neurochemistry 66:1617-1624.

33. De Vries, D D, Went, L N, Bruyn, G W, Scholte, H R, Hofstra, R M, Bolhuis, P A, van Oost, B A, Jun. A S, Trounce, I A, Brown, M D, Shoffner, J M, and Wallace, D C. 1996. Genetic and biochemical impairment of mitochondrial complex I activity in a family with Leber hereditary optic neuropathy and hereditary spastic dystoniaAm J. Hum Genet 58:703-11.

34. Dixon, E P, Stephenson, D T, Clemens, J A, and Little, S P. 1997. Bcl-X-short is elevated following severe global ischemia in rat brains. Brain Research, 776:222-229.

35. Du, Y S, Bales, K R, Dodel, R C, HamiltonByrd, E, Horn, J W, Czilli, D L, Simmons, L K, Ni, B H, and Paul, S M. 1997. Activation of a caspase 3-related cysteine protease is required for glutamate-mediated apoptosis of cultured cerebellar granule neurons. PNAS 94:11657-11662.

36. Du, Y S, Dodel, R C, Bales, K R, Jemmerson, R, HamiltonByrd, E, and Paul, S M. 1997. Involvement of a caspase-3-like cysteine protease in 1-methyl-4-phenylpyridinium-mediated apoptosis of cultured cerebellar granule neurons. J. Neurochemistry 69:1382-1388.

37. Evans, W H, Carlile, G, Rahrran, S, and Torok, K. 1992. Gap junction communication channel: peptides and anti-peptide antibodies as structural probes. Biochem Soc Trans 20:856-61.

38. Ferrari, G, Yan, C Y, and Greene, L A. 1995. N-acetylcysteine (D- and L-stereoisomers) prevents apoptotic death of neuronal cells. J. Neurosci 15:2857-66.

39. Freeman, R S, Estus, S, and Johnson, E M, Jr. 1994. Analysis of cell cycle-related gene expression in

postmitotic neurons: selective induction of Cyclin DI during programmed cell death. Neuron 12:343-55.

40. Gabellieri, E, RahuelClermont, S, Braniant, G, and Strambini, G B. 1996. Effects of NAD(+) binding on the luminescence of tryptophans 84 and 310 of glyceraldehyde-3-phosphate dehydrogenase from Bacillus stearothermophilus. Biochemistry 35:12549-12559.

41. Ghelli, A, Benelli, B, and Esposti, M D. 1997. Measurement of the membrane potential generated by complex I in submitochondrial particles. J. Biochemistry 121:746-755.

42. Gillardon, F, Wickert, H, and Zimmermann, M. 1995. Up-regulation of bax and downregulation of bc1-2 is associated with kainate-induced apoptosis in mouse brain. Neurosci Lett 192:85-8.

43. Greeniund, L J S, Deckwerth, T L, and Johnson, E M. 1995. Superoxide Dismutase Delays Neuronal Apoptosis: A Role for Reactive Oxygen Species in Programmed Neuronal Death. Neuron 14:303-315.

44. Greenwood, C E, Tatton, W G, Seniak, N A, and Biddle, F E. 1991. Increased dopamine synthesis in aging substantia nigra neurons. Neurobiol. Aging 12(5):557-565.

45. Gressner, A M, Lahme, B, and Roth, S. 1997. Attenuation of TGF-beta-induced apoptosis in primary cultures of hepatocytes by calpain inhibitors. Biochem. Biophys Res Comm 231:457-462.

46. Haas, R H, Nasirian, F, Nakano, K, Ward, D, Pay, M, Hill, R, and Shults, C W. 1995. Low platelet mitochondrial complex I and complex 11/lil activity in early untreated Parkinson's disease. Ann Neurol 377:714-22.

47. Hara, H, Friedlander, R M, Gagliardini, V, Ayata, C, Fink, K, Huang, Z H, ShimizuSasamata, M, Yuan, J Y, and Moskowitz, M A. 1997. Inhibition of interleukin 1 beta converting enzyme family proteases reduces ischemic and excitotoxic neuronal damage. PNAS 94:2007-2012.

48. Hassouna, I, Wickert, H, Zimmermann, M, and Gillardon, F. 1996. Increase in bax expression in substantia nigra following 1-methyl-4-phenyl-1,2,3,6-tetrahydropyridine (MPTP) treatment of mice. Neuroscience Letters 204:85-88.

49. Hatefi, Y, and Rieske, J S. 1967. Preparation and properties of DPNH-coenzyme Q reductase. Methods Enzymology 10:235-239.

50. Hattori, N, Tanaka, M, Ozawa, T, and Mizuno, Y. 1991. Immunohistochemical studies on complexes 1, I1,111, and IV of mitochondria in Parkinson's disease. Ann Neurol 30:563-71.

51. Haupt, Y, Barak, Y, and Oren, M. 1996. Cell type specific inhibition of p53 mediated apoptosis by mdm2. EMBO J. 15:1598-1606.

52. Hayashi, J, Takemitsu, M, Goto, Y, and Nonaka, I. 1994. Human mitochondria and mitochondrial genome function as a single dynamic cellular unit. J. Cell Biol 125:43-50.

53. Hennet, T G, Bertoni, G, Richter, C, and Peterhans, E. 1993. Expression of Bc 1-2 protein enhances the survival of mouse fibrosarcoid cells in tumor necrosis factor-mediated cytotoxicity. CancerRes. 53:1456-1460.

54. Hirsch, T, Marzo, I, and Kroemer, G. 1997. Role of the mitochondrial permeability transition pore in apoptosis. Bioscience Reports 17:67-76.

55. Ho, Y S, Lee, H M, Mou, T C, Wang, Y J, and Lin, J K. 1997. Suppression of nitric oxideinduced apoptosis by N-acetyl-L-cysteine through modulation of glutathione, bc 1-2, and bax protein levels. Molecular Carcinogenesis 19:101-113.

56. Hsu, Y T, Wolter, K G, and Youle, R J. 1997. Cytosol-to-membrane redistribution of Bax and Bcl-X-L during apoptosis. PNAS 94:3668-3672.

57. Huby, R D, Carlile, G W, and Ley, S C. 1995. Interactions between the protein-tyrosine kinase ZAP-70, the proto-oncoprotein Vav, and tubulin in Jurkat T cells. J Biol Chem 270:30241-4.

58. Ishibashi, Y, Nishimaki, K, Asoh, S, NanbuWakao, R, Vamada, T, and Ohta, S. 1998. Pore formation domain of human pro-apoptotic bax induces mammalian apoptosis as well as bacterial death without antagonizing anti-apoptotic factors. Biochem Biophys Res Comm 243:609-616.

59. Ishitani, R, and Chuang, D M. 1996. Glyceraldehyde-3-phosphate dehydrogenase antisense oligodeoxynucleotides protect against cytosine arabinonucleoside-induced apoptosis in cultured cerebellar neurons. PNAS 93:9937-9941.

60. Itoga, M, Tsuchlya, M, Ishino, H, and Shimoyama, M. 1997. Nitric oxide-induced modification of glyceraldehyde-3-phosphate dehydrogenase with NAD(+) is not ADP-ribosylation. J Biochem 121:1041-1046.

61. Jordan, J, Galindo, M F, and Miller, R J. 1997. Role of calpain- and interleukin-1 beta converting enzyme

like proteases in the beta-amyloid-induced death of rat hippocampal neurons in culture. J Neurochem 68:16121621.

62. Jordan, J, Galindo, M F, Prehn, J H M, Weichselbaum, R R, Beckett, M, Ghadge, G D, Roos, R P, Leiden, J M, and Miller, R J. 1997. P53 expression induces apoptosis in hippocampal pyramidal neuron cultures. J. Neuroscience 17:1397-1405.

63. Ju, W Y, Holland, D P, and Tatton, W G. 1994. (−)-Deprenyl alters the time course of death of axotomized facial motoneurons and the hypertrophy of neighboring astrocytes in immature rats. Exp Neurol 126:233-46.

64. Kantrow, S P, and Piantadosi, C A. 1997. Release of cytochrome c from liver mitochondria during permeability transition. Biochem Biophys Res Comm 232:669-671.

65. Kim, H, Feil, I K, Verlinde, C L M J, Petra, P H, and Hol, W G 1995. Crystal sructure of glycosomal glyceraldehyde 3-phosphate dehydrogenase from Leishmania mexicana:Implications for structure based drug design and a new position for the inorganic phosphate binding site. Biochemistry 34:14975-14986.

66. King, M P, and Attardi, G. 1989. Human cells lacking mtDNA: repopulation with exogenous mitochondria by complementation. Science 246:500-503.

67. King, T E. 1967. Preparation of succinate cytochrome c reductase and the cytochrome b-cl particle, and reconstitution of succinate cytochrome c reductase. Methods Enzymology 10:216-225.

68. Kluck, R M, BossyWetzel, E, Green, D R, and Newmeyer, D D. 1997. The release of cytochrome c from mitochondria: A primary site for Bc 1-2 regulation of apoptosis. Science 275:1132-1136.

69. Korsmeyer, S J, Yin, X M, Oltvai, Z N, Veis Novack, D J, and Linette, G P. 1995. Reactive oxygen species a-d the regulation of cell death by the Bc 1-2 gene family. Biochim Biophys Acta 1271:63-6.

7,6 i 1<,osel, S, Egensperger, R, vonEiken, U, Mehraein, P, and Graeber, M B. 1997. On the question of i,˜ptosis˜n the parkinsonian substantia nigra. Acta Neuropathologica 93:105-108.

71. Kr˜gten, E, Lalande, I, Zimmermann, K, Roggo, S, Schindler, P, Muller, D, van Oostrum, J, Waldmeier, P, and Fur,it, P. 1998. Glyceraldehyde-3-phosphate Dehydrogenase, the Putative Target of the Antiapoptotic Compounds CGP 3466 and R-(−)-Deprenyl. J Biol Chem 273:5821-5828.

72. Krige, D, Carroll, M T, Cooper, J M, Marsden, C D, and Schapira, A H. 1992. Platelet mitochondrial function in Parkinson's disease. Ann Neurol 32:782-8.

73. Kroemer, G. 1997. The proto-oncogene Bc 1-2 and its role in regulating apoptosis. Nature Medicine 3:614-620.

74. Lestienne, P, Nelson, J, Riederer, P, Jellinger, K, and Reichmann, H. 1990. Normal mitochondrial genome in brain from patients with Parkinson's disease and complex I defect [published erratum appears in J. Neurochem April 1991; 56(4):1457]. J Neurochem 55:1810-2.

75. Li, K, Neofer, P D, and Williams, R S. 1995. Nuclear responses to depletion of mitochondrial DNA in human cells. Am J Physiol 269:C1265-70.

76. Litzkas, P, Jha, K K, and Ozer, H L. 1984. Efficient transfer of cloned DNA into human diploid cells: protoplast fusion in suspension. Mol Cell Biol 4:2549-52.

77. Liu, X S, Kim, C N, Yang, J, Jemmerson, R, and Wang, X D. 1996. Induction of apoptotic program in cellfree extracts: Requirement for dATP and cytochrome c. Cell 86:147-157.

78. Lucking, C B, Kosel, S, Mehraein, P, and Graeber, M B. 1995. Absence of the mitochondrial A7237T mutation in Parkinson's disease. Biochem Biophys Res Commun 211:700˜.

79. MacGibbon, G A, Lawlor, P A, Sirimanne, E S, Walton, M R, Connor, B, Young, D, Williams, C, Gluckman, P, Faull, R L M, Hughes, P, and Dragunow, M. 1997. Bax expression in mammalian neurons undergoing apoptosis, and in Alzheimer's disease hippocampus. Brain Research 750:223-234.

80. Macleod, K F, Hu, Y, and Jacks, T. 1996. Loss of Rb activates both p53-dependent and independent cell death pathways in the developing mouse nervous system. Embo J. 15:617-888.

81. Mann, V M, Cooper, J M, Daniel, S E, Srai, K, Jenner, P, and Marsden, C D. 1994. Complex I, iron and ferritin in Parkinson's disease substantia nigra. Ann Neurol 36:876-881.

82. Mann, V M, Cooper, J M, Krige, D, Daniel, S E, Schapira, A H, and Marsden, C D. 1992. Brain, skeletal muscle and platelet homogenate mitochondrial function in Parkinson's disease. Brain 115:333-2.

83. Marchetti, P, Castedo, M, Susin, S A, Zamzami, N, Hirsch, T, Macho, A, Haeffner, A, Hirsch, F, Geuskens, M, and Kroemer, G. 1996. Mitochondrial permeability transition is a central coordinating event of apoptosis. J Exp Med 184:1155-60.

84. Marchetti, P, Hirsch, T, Zamzami, N, Castedo, M, Decaudin, D, Susin, S A, Masse, B, and Kroemer, G. 1996. Mitochondrial permeability transition triggers lymphocyte apoptosis. J Immunology 157:4830-836.

85. Marchetti, P, Susin, S A, Decaudin, D, Gamen, S, Castedo, M, Hirsch, T, Zamzami, N, Naval, J, Senik, A, and Kroemer, G. 1996. Apoptosis-associated derangement of mitochondrial function in cells lacking mitochondrial DNA. Cancer Research 56:2033-2038.

86. Marin, P, Maus, M, Bockaert, J, Glowinski, J, and Premont, J. 1995. Oxygen free radicals enhance the nitric oxide-induced covalent NAD(+)-linkage to neuronal glyceraldehyde-3-phosphate dehydrogenase. Biochemical Journal 309:891-898.

87. McCarthy, M J, Rubin, L L, and Philpott, K L. 1997. Involvement of caspases in sympathetic neuron apoptosis. Journal of Cell Science 110:2165-2173.

88. Merrick, B A, He, C, Witcher, L L, Patterson, R M, Reid, J J, Pence Pawlowski, P M, and Selkirk, J K. 1996. HSP binding and mitochondrial localization of p53 protein in human HT1080 and mouse C3H10T1/2 cell lines. Biochim Biophys Acta 1297:57-68.

89. Merry, D E, and Korsmeyer, S J. 1997. Bc1-2 gene family in the nervous system. Ann Rev Neuroscience 20:245-267.

90. Messmer, U K, and Brune, B. 1996. Modification of macrophage glyceraldehyde-3-phosphate dehydrogenase in response to nitric oxide. Eur J Pharmacol 302:171-182.

91. Minton, A P, and Wilf, J. 1981. Effect of macromolecular cowding upon structure formation and function of an enzyme: glyceraldehyde 3-phosphate dehydrogenase. Biochemistry 20:4821-826.

92. Mitry, R R, Sarraf, C E, Wu, C G, Pignatelli, M, and Habib, N A. 1997. Wild-type p53 induces apoptosis in Hep3B through up-regulation of bax expression. Laboratory Investigation 77:369-378.

93. Mizuno, Y, Ikebe, S, Hattori, N, Nakagawa Hattori, Y, Mochizuki, H, Tanaka, M, and Ozawa, T. 1995. Role of mitochondria in the etiology and pathogenesis of Parkinson's disease. Biochim BiophysActa 1271:26574.

94. Mizuno, Y, Matuda, S, Yoshino, H, Mori, H, Hattori, N, and Ikebe, S. 1994. An immunohistochemical study on alpha-ketoglutarate dehydrogenase complex in Parkinson's disease. Ann Neurol 35:204-10.

95. Mizuno, Y, Ohta, S, Tanaka, M, Takamiya, S, Suzuki, K, sato, T, Oya, H, Ozawa, T, and Kagawa, Y. 1989. Deficiencies in complex I subunits of the respiratory chain in Parkinson's disease. Biochem. Biophys. Res. Comm. 163:1450-1455.

96. Mochizuki, H, Goto, K, Mori, H, and Mizuno, Y. 1996. Histochemical detection of apoptosis in Parkinson's disease. J Neurological Scis 137:120-123.

97. Mogi, M, Harada, M, Kondo, T, Mizuno, Y, Narabayashi, H, Riederer, P, and Nagatsu, T. 1996. bc1-2 Protein is increased in the brain from parkinsonian patients. Neuroscience Letters 215:137-139.

98. Mogi, M, Harada, M, Kondo, T, Mizuno, Y, Narabayashi, H, Riederer, P, and Nagatsu, T. 1996. The soluble form of Fas molecule is elevated in parkinsonian brain tissues. Neurosci Leff 220:195-8.

99. Monaghan, P, Perusinghe, N, Carlile, G, and Evans, W H. 1994. Rapid modulation of gap junction expression in mouse mammary gland during pregnancy, lactation, and involution. J Histochem Cytochem 42:931-8.

100. Mullis, K, Faloona, F, Scharf, S, Saiki, R, Horn, G, and Erlich, H. 1986. Specific enzymatic amplification of DNA in vitro: the polymerase chain reaction. Cold Spring Harbour Symposium 51:263-273.

101. Mytilineou, C, Werner, P, Molinari, S, DiRocco, A, Cohen, G, and Yahr, M D. 1994. Impaired oxidative decarboxylation of pyruvate in fibroblasts from patients with Parkinson's disease. J Neural Trans Parkinsons Disease and Dementia Section 8:223-228.

102. Nagy, E, and Rigby, W F C. 1995. Glyceraldehyde-3-phosphate dehydrogenase selectively binds AU-rich RNA in the NAD(+)-binding region (Rossmann fold). J. Biological Chemistry 270:2755-2763.

103. Nakazawa, M, Uehara, T, and Nomura, Y. 1997. Koningic acid (a potent glyceraldehyde-3-phosphate dehydrogenase inhibitor) induced fragmentation and condensation of DNA in NG108-15 cells. J. Neurochemistry 68:2493-2499.

104. Nath, R, Raser, K J, McGinnis, K, Nadimpalli, R, Stafford, D, and Wang, K K W. 1996. Effects of ICE-like protease and calpain inhibitors on neuronal apoptosis. Neuroreport 8:249-255.

105. Neupert, W. 1997. Protein import into mitochondria. Annu Rev Biochem 66:863-917.

106. Newmeyer, D D, Farschon, D M, and Reed, J C. 1994. Cell-free apoptosis in Xenopus egg extracts: inhibition by Bc1-2 and requirement for an organelle fraction enriched in mitochondria Cell 79:353-64.

107. Oberhammer, F, Wilson, J W, Dive, C, Morris, I D, Hickman, J A, Wakeling, A E, Walker, P R, and Sikorska, M. 1993. Apoptotic death in epithelial cells: cleavage of DNA to 300 and/or 50 kb fragments prior to and or in the absence of internucleosomal fragmentation. EMBO J. 12:3679-3684.

108. Olanow, C W, and Tatton, W G. 1998. Etiology And Pathogenesis Of Parkinson's disease. Ann. Rev. Neurosci. In Press.

109. Pandey, S, Walker, P R, and Sikorska, M. 1994. Separate pools of endonuclease activity are responsible for intemucleosomal and high molecular mass DNA fragmentation during apoptosis. Biochem Cell Biol 72:625-9.

110. Pandol, S J, Fitzsimmons, T, Schoeffield Payne, M, Carlile, G W, and Evans, W H. 1995. Isolation of subcellular agonist-sensitive calcium stores from the pancreatic acinar cell. Cell Calcium 18:364-76.

111. Parker, W D, Boyson, S J, and Parks, J K. 1989. Abnormalities of electron transport in Parkinson's disease. Ann. Neurol. 50:719-723.

112. Polyak, K, Xia, Y, Zweier, J L, Kinzler, K W, and Vogelstein, B. 1997. A model for p53-induced apoptosis. Nature: 300-305.

113. Przedborski, S, Kostic, V, Jacksonlewis, V, Naini, A B, Simonetti, S, Fahn, S, Carlson, E, Epstein, C J, and Cadet, J L. 1992. Transgenic Mice with Increased Cu/Zn-Superoxide Dismutase Activity Are Resistant to Nmethyl4-phenyl-1,2,3,6-tetrahydropyridine-induced Neurotoxicity. J. Neuroscience 12:1658-1667.

114. Rabizadeh, S, Gralla, E B, Borchelt, D R, Gwinn, R, Valentine, J S, Sisodia, S, Wong, P, Lee, M, Hahn, H, and Bredesen, D E. 1995. Mutations associated with amyotrophic lateral sclerosis convert superoxide dismutase from an antiapoptotic gene to a proapoptotic gene: studies in yeast and neural cells. PNAs 92:30248.

115. Ragan, C l, Wilson, M T, V. M., D- U, and Lowe, P N. 1987. Subfractionation of mitochondria and isolation of the proteins of oxidative phosphorylation. Pages 79-112 in V. M. Darley-Usmar, D. Rickwood, and M. T. Wilson, Eds., Mitochondria, A Practical Approach. IRL Press, London.

116. Reichmann, H, Naumann, M, Hauck, S. and Janetzky, B. 1994. Respiratory chain and mitochondrial deoxyribonucleic acid in blood cells from patients with focal and generalized dystonia. Mov Disord 9:597-600.

117. Roise, D. 1997. Recognition and binding of mitochondrial presequences during the import of proteins into mitochondria. J. Bioenerg Biomembr 29:19-27.

118. Rothstein, J D, Bristol, L A, Hosler, B, Brown, R H, Jr., and Kuncl, R W. 1994. Chronic inhibition of superoxide dismutase produces apoptotic death of spinal neurons. PNAS 91:415-59.

119. Salmeron, A, Ahmad, T B, Carlile, G W, Pappin, D, Narsimhan, R P, and Ley, S C. 1996. Activation of MEK-1 and SEK-1 by Tp1-2 proto-oncoprotein, a novel MAP kinase kinase kinase. Embo J. 15:817-26.

120. Sambrook, J, Fritsch, E F, and Maniatis, T. 1989. Molecular Cloning: A Laboratory Manual, 2nd ed. Cold Spring Harbor Lab. Press, Plainview, N.Y.

121. Saunders, P A, ChaleckaFranaszek, E, and Chuang, D M. 1997. Subcellular distribution of glyceraldehyde-3-phosphate dehydrogenase in cerebellar granule cells undergoing cytosine arabinoside-induced apoptosis. J. Neurochem. 69:1820-1828.

122. Sawa, A, Khan, M, Hester, L D, and Snyder, S H. 1997. Glyceraldehyde-3-phosphate dehydrogenase: Nuclear translocation participates in neuronal and nonneuronal cell death. PNAS 94:11669-11674.

123. Scarpulla, R C. 1997. Nuclear control of respiratory chain expression in mammalian cells. J BioenergBiomembr 29:109-119.

124. Schapira, A H. 1995. Nuclear and mitochondrial genetics in Parkinson's disease. J. Med Genet 32:4114.

125. Schapira, A H, Cooper, J M, Dexter, D, Clark, J B, Jenner, P, and Marsden, C D. 1990. Mitochondrial complex I deficiency in Parkinson's disease. J. Neurochem 54:823-7.

126. Schapira, A H, Mann, V M, Cooper, J M, Dexter, D, Daniel, S E, Jenner, P, Clark, J B, and Marsden, C D. 1990. Anatomic and disease specificity of NADH CoQI reductase (complex 1) deficiency in Parkinson's disease. J Neurochem 55:2142-5.

127. Scorrano, L, Nicolli, A, Basso, E, Petronilli, V, and Bernardi, P. 1997. Two modes of activation of the permeability transition pore: The role of mitochondrial cyclophilin. Mol Cell Biochem 174:181-184.

128. Scorrano, L, Petronilli, V, and Bernardi, P. 1997. On the voltage dependence of the mitochondrial permeability transition pore—A critical appraisal. J Biological Chem 272:12295-12299.

129. Seniuk, N A, Altares, M, Dunn, R, and Richardson, P M. 1992. Decreased synthesis of ciliary neurotrophic factor in degenerating peripheral nerves. Brain Res. 572:300-302.

130. Seniuk, N A, Henderson, J T, Tatton, W G, and Roder, J C. 1994. Increased CNTF gene expression in process-bearing astrocytes following injury is augmented by R(−)-deprenyl. J Neurosci Res 37:278-86.

131. Seniuk, N A, Tatton, W G, Cannon, P, Garber, A, and Dixon, G. 1991. First expression of protamine message in trout testis. Proc. MY Acad. Sci. 637:277-288

132. Seniuk, N A, Tatton, W G, and Greenwood, C E. 1990. Dose-dependent destruction of the coeruleuscortical and nigral-striatal projections by MPTP. Brain Res. 527:7-20.

133. Seniuk-Tatton, N A, Henderson, J T, and Roder, J C. 1995. Neurons express ciliary neurotrophic factor mRNA in the early postnatal and adult rat brain. J. Neurosci. Res. 41:663-676.

134. Sherratt, H S A. 1991. Mitochondria: structure and function. Rev. Neurol. (Paris J 147:417-430.

135. Sirover, M A. 1997. Role of the glycolytic protein, glyceraldehyde-3-phosphate dehydrogenase, in normal cell function and in cell pathology. J Cell Biochemist˜y66:133-140.

136. Smith, D B, and Johnson, K S. 1988. Single step purification of polypeptides expressed in Escherichia coli as fusions with glutathione transferase. Gene 67:31-40.

137. Sugrue, M M, Shin, D Y, Lee, S W, and Aaronson, S A. 1997. Wild-type p53 triggers a rapid senescence program in human tumor cells lacking functional p53. PNAS 94:9648-53.

138. Sunaga, K, Takahashi, H, Chuang, D M, and Ishitani, R. 1995. Glyceraldehyde-3-phosphate dehydrogenase is over-expressed during apoptotic death of neuronal cultures and is recognized by a monoclonal antibody against amyloid plaques from Alzheimer's brain. Neuroscience Leffers 200:133-136.

139. Susin, S A, Zamzami, N, Castedo, M, Daugas, E, Wang, H G, Geley, S, Fassy, F, Reed, J C, and Kroemer, G. 1997. The central executioner of apoptosis: Multiple connections between protease activation and mitochondria in Fas/APO-1/CD95- and ceramide-induced apoptosis. J Experimental Medicine 186:25-37.

140. Susin, S A, Zamzami, N, Castedo, M, Hirsch, T, Marchetti, P, Macho, A, Daugas, E, Geuskens, M, and Kroemer, G. 1996. Bc 1-2 inhibits the mitochondrial release of an apoptogenic protease. J. Experimental Medicine 184:1331-1341.

141. Susin, S A, Zamzami, N, and Kroemer, G. 1996. The cell biology of apoptosis: evidence for the implication of mitochondria. Apoptosis 1:231-242.

142. Swerdlow, R H, Parks, J K, Miller, S W, Tuttle, J B, Trimmer, P A, Sheehan, J P, Bennett, J P, Jr., Davis, R E, and Parker, W D, Jr. 1996. Origin and functional consequences of the complex I defect in Parkinson's disease. Ann Neurol 40:663-71.

143. Tateyama, H, Tada, T, Hattori, H, Murase, T, Li, W X, and Eimoto, T. 1998. Effects of prefixation and fixation times on apoptosis detection by in situ end-labeling of fragmented DNA. ArchPath & Lab Medicine 122:252-255.

144. Tatton, N A, and Kish, S J. 1997. In situ detection of apoptotic nuclei in the substantia nigra compacta of 1-methyl-4-phenyl-1,2,3,6-tetrahydropyridine-treated mice using terminal deoxynucleotidyl transferase labelling and acridine orange staining. Neurosci 77:1037-1048.

145. Tatton, N A, Maclean-Fraser, A, Tatton, W G, Perl, D F, and Olanow, C W. 1998. A Fluorescent DoubleLabeling Method To Detect And Confirm Apoptotic Nuclei In Parkinson's disease. Ann. Neurol. In Press.

146. Tatton, W, Ju, W, Wadia, J, Ansari, K, Zhang, F, Buys, Y, and Seniuk, N. 1994. (−)-Deprenyl reduces neuronal apoptosis by maintaining Bc1-2 synthesis and mitochondrial membrane potential. Move Disord 9:4.

147. Tatton, W G, and ChalmersRedman, R M E. 1996. Modulation of gene expression rather than monoamine oxidase inhibition: (−)-deprenyl-related compounds in controlling neurodegeneration. Neuro/47:S171-S183.

148. Tatton, W G, and Greenwood, C E. 1991. Rescue of Dying Neurons: A New Action For Deprenyl In MPTP Parkinsonism. J. Neurosc. Res. 30:666-627.

149. Tatton, W G, Greenwood, C E, Salo, P. and Seniuk, N A. 1991. Transmitter synthesis increases in substantia nigra neurons in aged mice. Neurosc. Letters 131:179-182.

150. Tatton, W G, Ju, W J H, Wadia, J, and Tatton, N A. 1996. Reduction of neuronal apoptosis by smali molecules: promise for new approaches to neurological therapy. Pages 209-229 in W. Olanow, M. Youdim, and P. Jenner, Eds., Neuroprotection and Neurodegeneration. Academic Press Ltd., New York.

151. Tatton, W G, Ju, W Y, Holiand, D P, Tai, C, and Kwan, M. 1994. (−)-Deprenyl reduces PC12 cell apoptosis by inducing new protein synthesis. J. Neurochem 63:1572-5.

152. Tatton, W G, and Olanow, C W. 1998. Apoptosis In Neurodegenerative Diseases: Role of Mitochondria. Biochime and Biophysica Acta In Press.

153. Tiranti, V, Munaro, M, Sandona, D, Lamantea, E, Rimoldi, M, DiDonato, S, Bisson, R, and Zeviani, M. 1995. Nuclear DNA origin of cytochrome c oxidase deficiency in Leigh's syndrome: genetic evidence based on patient's-derived rho degrees transformants. Hum Mol Genet 4:2017-2023.

154. Tompkins, M M, and Hill, W D. 1997. Contribution of somal Lewy bodies to neuronal death. Brain Res 775:24-9.

155. Troy, C M, and Shelanski, M L. 1994. Down-regulation of copper/zinc superoxide dismutase causes apoptotic death in PC12 neuronal cells. PNAS 91:6384-7.

156. Tsuda, T, Munthasser, S, Fraser, P E, Percy, M E, Rainero, I, Vaula, G, Pinessi, L, Bergamini, L, Vignocchi, G, Crapper McLachlan, D R, Tatton, W G, and St. George-Hyslop, P. 1994. Analysis of the Functional Effects of a Mutation in SODI Associated with Familial Amyotrophic Lateral Sclerosis. Neuron 13:727-736.

157. Villa, P, Kaufmann, S H, and Eamshaw, W C. 1997. Caspases and caspase inhibitors. Trends in Biochemical Sciences 22:388-393.

158. Vyas, S, JavoyAgid, F, Herrero, M T, Strada, O. Boissiere, F, Hibner, U, and Agid, Y. 1997. Expression of bc1-2 in adult human brain regions with special reference to neurodegenerative disorders. J. Neurochemistry 69:223-231.

159. Wadia, J S, Chalmers-Redman, R M E, Ju, W J H, Carlile, G W, Phillips, J L, and Tatton, W G. 1998. Mitochondrial Membrane Potential And Nuclear Changes In Apoptosis Caused By Trophic Withdrawal: Time Course And Modification By (−)-Deprenyl. J. Neurosci. 18:932-947.

160. Wagner, A J, Kokontis, J M, and Hay, N. 1994. MYC-mediated apoptosisnrequires wild type p53 in a manner independent of cell cycle arrest and the ability of p53 to induce p21. Genes Dev. 8:2817-2830.

161. Wharton, D C, and Tzagoloff, A. 1967. Cytochrome oxidase from beef heart mitochondria. Methods Enzymology 10:245-250.

162. Wolter, K G, Hsu, Y T, Smith, C L, Nechushtan, A, Xi, X G, and Youle, R J. 1997. Movement of Bax from the cytosol to mitochondria during apoptosis. J. Cell Biology 139:1281-1292.

163. Xiang, H, Kinoshita, Y, Knudson, C M, Korsmeyer, S J, Schwartzkroin, P A, and Morrison, R S. 1998. Bax involvement in p53-mediated neuronal cell death. J. Neuroscience 18:1363-1373.

164. Yan, C Y, Ferrari, G, and Greene, L A. 1995. N-acetylcysteine-promoted survival of PC12 cells is glutathione-independent but transcription-dependent. J Biol Chem 270:26827-32.

165. Yang, J, Liu, X, Bhalla, K, Kim, C N, Ibrado, A M, Cai, J. Peng, T l, Jones, D P, and Wang, X. 1997. Prevention of apoptosis by Bc1-2: release of cytochrome c from mitochondria blocked Science 275:1129-32.

166. Yoneda, M, Katsumata, K, Hayakawa, M, Tanaka, M, and Ozawa, T. 1995. Oxygen stress induces an apoptotic cell death associated with fragmentation of mitochondrial genome. Biochem Biophys Res Commun 209:723-9.

167. Yoshino, H, Nakagawa Hattori, Y, Kondo, T, and Miztno, Y. 1992. Mitochondrial complex I and II activities of lymphocytes and platelets in Parkinsonis disease. J Neural Transm Park Dis Dement Sect 4:27-34.

168. Zamzami, N, Marchetti, P, Castedo, M, Hirsh, T, Susin, S A, Masse, B, and Kroemer, G. 1996. Inhibitors of permeability transition interfere with the disruption of mitochondrial transmembrane potential during apoptosis. FEBS Letters 384:53-57.

169. Zhang, J. 1997. Use of biotinylated NAD to label and purify ADP-ribosylated proteins. Pages 255-265 in D. B. Mccormick, J. W. Suttie, and C. Wagner, Eds., Vitamins and Coenzymes, Pt J. Academic Press Inc, 525 B StreeVSuite 1900/San Diego/Calif. 921014495.

170. Zhang, J, and Snyder, S H. 1995. Nitric oxide in the nervous system. Annual Review of Pharmacology and Toxicology 35:213-233.

171. Zoratti, M, and Szabo, I. 1995. The mitochondrial permeability transition. Biochimica et Biophysica Acta Reviews on Biomembranes 1241:139-176.

What is claimed is:

1. A method for identifying a dehydrogenase oligomeric modulator, comprising:
contacting the dehydrogenase with a compound under conditions which allow oligomerization of the dehydrogenase to occur; and
measuring the ability of the compound to modulate oligomerization of the dehydrogenase as an indication of whether the compound is a dehydrogenase oligomeric modulator.

2. A method for identifying an oligomeric modulator of GAPDH, comprising:
contacting GAPDH with a compound under conditions which allow oligomerization of GAPDH to occur; and
measuring the ability of the compound to modulate oligomerization of GAPDH as an indication of whether the compound is an oligomeric modulator of GAPDH.

3. The method of claim 2 wherein the ability of the compound to modulate oligomerization of GAPDH is its ability to prevent the formation of a tetramer of GAPDH.

4. The method of claim 2 wherein the ability of the compound to modulate oligomerization of GAPDH is its ability to enhance the formation of a dimer of GAPDH.

5. A method for identifying an antiapoptotic agent, comprising:
contacting GAPDH with a compound under conditions which allow oligomerization of GAPDH to occur; and
measuring the ability of the compound to modulate oligomerization of GAPDH as an indication of whether the compound is an antiapoptotic agent.

6. The method of claim 5 wherein the ability of the compound to modulate oligomerization of GAPDH is its ability to prevent the formation of a tetramer of GAPDH.

7. The method of claim 5 wherein the ability of the compound to modulate oligomerization of GAPDH is its ability to enhance the formation of a dimer of GAPDH.

8. A method of screening a library of compounds for their ability to modulate oligomerization of GAPDH, comprising:
contacting GAPDH with a sample of a library of compounds under conditions which allow oligomerization of GAPDH to occur;
classifying any oligomers formed; and
identifying the compounds that modulate oligomerization of GAPDH as an indication of whether the compounds are modulators of oligomerization of GAPDH.

9. The method of claim 8 wherein the ability of the identified compounds to modulate oligomerization of GAPDH is the ability to prevent the formation of a tetramer of GAPDH.

10. The method of claim 8 wherein the ability of the identified compounds to modulate oligomerization of GAPDH is the ability to enhance the formation of a dimer of GAPDH.

11. The method of claim 8 wherein the ability of the identified compound to modulate oligomerization of GAPDH is the ability to bind to GAPDH and thus prevent GAPDH oligomer formation.

12. A method for identifying an antiapoptotic agent, comprising:
contacting a dehydrogenase with a compound under conditions which allow oligomerization of the dehydrogenase to occur; and
measuring the ability of the compound to modulate oligomerization of the dehydrogenase as an indication of whether the compound is an antiapoptotic agent.

13. A method of screening a library of compounds for their ability to modulate dehydrogenase oligomerization, comprising:
contacting a dehydrogenase with a sample of a library of compounds under conditions which allow oligomerization of the dehydrogenase to occur;
classifying any oligomers formed; and
identifying the compounds that modulate dehydrogenase oligomerization as an indication of whether the compounds are dehydrogenase oligomeric modulators.

14. The method of claim 13 wherein the ability of the identified compound to modulate dehydrogenase oligomerization is the ability to bind to the dehydrogenase and thus prevent the dehydrogenase oligomer formation.
